(19) United States
(12) Patent Application Publication
(10) Pub. No.: US 2014/0031583 A1
(43) Pub. Date: Jan. 30, 2014

(54) OLEFIN CONDITIONING IN A FAST CATALYTIC PYROLYSIS RECYCLE PROCESS

(71) Applicant: Anellotech, Inc., Pearl River, NY (US)

(72) Inventors: Terry Mazanec, Solon, OH (US); Jeff Whiting, Goshen, VT (US); Fred Pesa, Aurora, OH (US); Greg Norenberg, Medina, OH (US)

(21) Appl. No.: 13/953,541

(22) Filed: Jul. 29, 2013

Related U.S. Application Data
(60) Provisional application No. 61/676,840, filed on Jul. 27, 2012.

(51) Int. Cl. C10G 1/00; C07C 41/06; C07C 51/14; C07C 37/00

(52) U.S. Cl. CPC C10G 1/002; C07C 37/00; C07C 41/06; C07C 51/14
USPC 562/521; 585/240; 568/840; 568/671

(57) ABSTRACT

This invention relates to improvements in the fast pyrolysis of biomass. In this invention, olefins are separated from the effluent stream of a pyrolysis reactor and at least a portion of the olefins are treated and the resulting treated stream recycled to the pyrolysis reactor for further conversion to valuable, useful products.

### RELATED APPLICATION

This application claims the benefit of priority U.S. provisional patent application Ser. No. 61/676,840, filed Jul. 27, 2012.

### INTRODUCTION

Due to its cost and wide availability, lignocellulosic biomass is under worldwide-study as a viable feedstock for renewable liquid biofuels and chemicals because of its low cost and wide availability. Biomass-derived fuels and chemicals substantially reduce net CO2 emissions if produced with minimal use of fossil fuels. However, lignocellulosic biomass is not currently commonly used as a liquid fuel or chemicals source because typical current conversion processes are not considered to be economically viable. Several routes are being examined to convert solid biomass to valuable liquid fuels and chemicals. At low temperatures (e.g., 200-260° C.) diesel range alkanes can be produced by a multi-step, aqueous-phase processing (APP) of aqueous carbohydrate solutions involving dehydration, aldol-condensation and dehydration/hydrogenation. However, APP requires that solid lignocellulosic biomass first be converted into aqueous carbohydrates. At higher temperatures (about 800° C.), solid biomass can be reformed to produce synthesis gas through partial oxidation over catalysts in an auto thermal, packed-bed reactor. The synthesis gas produced from this reaction can be fed to a secondary process such as Fischer-Tropsch or methanol synthesis to make fuels and chemicals. An ideal process for solid biomass conversion would involve the production of a liquid fuel that can be easily handled within existing infrastructure from solid biomass in a single step, at short residence times.

Unfortunately, neither the APP nor the syngas process meets such criteria. Another approach for biofuel production is fast pyrolysis, which can involve, for example, rapidly heating biomass (e.g., about 500° C./second) to intermediate temperatures (e.g., 400-600° C.) followed by rapid cooling (e.g., residence times of 1-2 seconds). (See, A. V. Bridgwater, Fast Pyrolysis of Biomass: A Handbook Volume 2, CPL Press, Newbury, UK, 2002.) Conventional fast pyrolysis often produces a thermally unstable, liquid product mixture, called bio-oil, an acidic, combustible liquid mixture of more than 300 compounds that degrades over time.

However, bio-oils are not compatible with existing liquid transportation fuels, such as gasoline and diesel, and their yields are low. Bio-oils require considerable further transformation to produce useful chemical intermediates as well. Accordingly, there remains an on-going search in the art for an economical, efficient route for the production of useful biofuels and related compounds from solid biomass.

Fast catalytic pyrolysis (FCP), developed by Professor George W. Huber at the University of Massachusetts Amherst, involves the conversion of biomass in a catalytic fluid-bed reactor to a mixture of aromatics, olefins, CO, CO2, char, ash, and a variety of water-soluble organics. See US App US20090227823 and WO2011031320A2 which are incorporated here by reference as if reproduced in full below. The aromatics include benzene, toluene, xylenes, and naphthalene (BTXN), among other aromatics. The olefins include ethylene (30-60% of olefins), propylene (30-50%), and lesser amounts of higher olefins. BTXN have high economic values and are easily transported, but C2-C4 olefins are not easily transported, so they are best produced where there is suitable infrastructure for their use; they are usually produced in massive quantities in purpose-built plants. Unfortunately, many biomass resources are not found near locations where olefins can be easily used. Thus a need exists for converting the olefins to additional aromatics in the FCP process.

Huber et al. in WO 2011/031320 describes a process as illustrated in FIG. 1.

As shown in FIG. 1, the product stream from reactor 20 passes into olefin recycler 102. The process has flexibility with respect to the positioning of the olefin recycler 102, as stated on page 23: “While optional product reactor 104 is shown located directly downstream of the reactor 20 in FIG. 1, it should be understood that the olefin recycler can be positioned at any point downstream of the reactor (e.g., reactor 20 in FIG. 1), and the reaction of olefins, aromatics, or other fluid hydrocarbon products are produced.” The recycle stream 100 is shown as being combined with the feed stream 10 upstream of dryer 14; however, it could alternatively be added before grinder 16 or fed directly back “to reactor 20, and/or combined with any of the catalyst stream (e.g., 34, 42, 44 [fluidizing stream], 46, and/or 47 [regenerated catalyst recycle stream].” The recycle stream is described as “comprising at least a portion of the separated olefins.

In Example 14, Huber et al. report that adding propylene to the reactor resulted in a higher yield of aromatics and lower yields of coke, carbon dioxide, and carbon monoxide. Similar results were reported in the thesis of Professor Huber's student, Torren Carlson, where he reported that increasing ethylene feed content slightly decreased aromatic and coke yields while carbon monoxide yield goes through a maximum at intermediate ethylene concentration.

Prior art processes suffer from the direct introduction of olefins into the FCP reactor at reaction conditions that are suitable for biomass pyrolysis, including temperatures above 300° C., and often above 500° C. Under such conditions, the olefins, particularly ethylene, are known to polymerize rapidly and form coke, an undesirable byproduct as one of the products. Methods that introduce olefins into the reactor that result in increased levels of incorporation of the olefins into the desired aromatic products are sought.

### SUMMARY OF THE INVENTION

In this invention, an olefin stream that is produced by pyrolysis of biomass is treated and at least a portion of the treated stream is returned to a pyrolysis reactor (typically the same reactor that produced the olefin stream). In various embodiments, the invention provides advantages, such as reducing char formation and/or increasing production of useful products such as BTXN.

In one aspect, the invention provides a method for producing one or more fluid hydrocarbon products from a hydrocarbonaceous material. In this method, a hydrocarbonaceous material is fed to a reactor, and at least a portion of the hydrocarbonaceous material is pyrolyzed within the reactor under reaction conditions sufficient to produce one or more pyrolysis products. The products are separated into at least two fractions, where at least one of the fractions comprises at least one olefin. For the sake of clarity, the portion that comprises at least one olefin is termed a “first fraction.” This terminology should not be interpreted to mean anything about the sequence in which the first fraction is separated from the other products. For example, the first fraction could be a gas stream that is separated from liquids that condense from the product stream, or the so-called first fraction could be derived by fractional distillation (or reactive distillation) of a condensed liquid fraction. In the broadest aspect of the invention, the only requirement of the first fraction is that it is derived (either as a direct product and/or a product of subsequent reactions) from the product stream of a biomass pyrolysis reaction and that it includes olefins. The first fraction may also contain alkynes. The first fraction is treated in a fashion that changes the chemical composition of the first fraction by adding a chemical compound and/or reacting at least some of the olefins in the first fraction to produce a treated fraction. The treated fraction is then added to a pyrolysis reactor, in some preferred embodiments, the treated fraction is recycled to the same reactor that produced the pyrolysis products mentioned above. The treatment could include, for example: hydrogenation, hydrolysis, hydroformylation, cyclization, dimerization, polymerization, alkylation, or other conversion process or combination of conversion processes. However, the invention is not limited to these treatments. In some embodiments, the treatment comprises one or any combination of the following steps: conversion of olefins to alcohols or ethers; addition of radical inhibiting agents; low temperature (80-400° C.) polymerization of olefins; reaction with CO to form carboxylic acids; alkylation to form alkylated aromatics, and hydrogenation of alkynes to olefins. For treatments involving chemical conversions, the first fraction can be passed into a reactor containing a catalyst selected for the desired chemical conversion.

In another embodiment a gaseous product stream is separated into an olefin poor and olefin rich stream and at least a portion of the olefin rich stream coming from an olefins separator is treated before mixing with the full recycle gas stream, thereby reducing the volume to be treated.

The invention also includes chemical systems comprising the apparatus and compositions described herein. The invention further includes the chemical compositions that occur as intermediates or final products that are described herein or that result from the methods described herein. For example, the invention includes pyrolysis products that additionally comprise radical inhibitors, or pyrolysis derivatives that comprise at least 1 mass % of C₅ to C₃₀ olefinic oligomers combined with a stream of low molecular weight olefins (such as ethylene and propylene) that would occur during or following a treatment in which an olefin stream was subjected to low temperature polymerization.

In the present invention, treatments are described that use ethylene, other olefins, or alkynes, or some combination of these to increase the yield of desired products. In some preferred embodiments, the conditioning of the olefins and/or alkynes by chemical treatment improves the incorporation of the carbon of the olefins and/or alkynes into desired aromatic products. The treatment of a stream that contains olefins, alkynes, or both olefins and alkynes, is referred to as olefin-conditioning, and the products of the treatment are referred to as olefin-conditioning products.

In some embodiments, one or more fluid hydrocarbon products may be produced from the pyrolysis products by dehydration, decarbonylation, decarboxylation, isomerization, oligomerization, and dehydrogenation reactions. In some preferred embodiments, pyrolysis and subsequent catalytic reaction processes occur in a single reactor. In some preferred embodiments, at least a portion of the olefins produced by the pyrolysis reaction and/or a subsequent reaction are treated and recycled into a feed stream, whereby the hydrocarbonaceous biomass material is fed to the pyrolysis reactor or combined with the fluidization fluid that is passed into a fluidized bed pyrolysis reactor.

In some embodiments, the feed composition (e.g., in feed stream 10 of FIG. 2) comprises a mixture of hydrocarbonaceous material and a catalyst. The mixture may comprise, for example, solids, liquids, and/or gases. In certain embodiments, the mixture comprises a composition of a solid catalyst and a solid hydrocarbonaceous material. In other embodiments, a catalyst may be provided separately from the reactor feed stream.

In some embodiments, one or more olefin conditioning products can be fed with the hydrocarbonaceous material and/or the catalyst. In FIG. 2 the olefin-conditioning step is shown as 80, with the olefin-conditioning products being fed with the fluidization gas. The olefin-conditioning process can be placed as shown in FIG. 2 after the compressor, 26, using a portion of the separated vapors 56 as the feed, or it can be placed downstream of compressor 26, or it can be fed as well by a portion of the vapor stream 33 that is separated from the solids in 32, or it can be fed by a portion of stream 60, or it can be fed by a portion of the vapors 74 that are separated from condensable components in 50, or some combination of these. The olefin-conditioning products may be of any suitable phase (solid, liquid, gas, or combinations thereof). The olefin-conditioning products and the hydrocarbonaceous material can be fed as part of the same stream as the hydrocarbonaceous material and/or the catalyst, or the olefin-conditioning products can be fed separately from the hydrocarbonaceous material and/or the catalyst. In some embodiments, the olefin-conditioning products may originate from one or more product streams from the reactor and/or a downstream process. Olefin-conditioning products may be reacted with hydrocarbonaceous material in any selected amount and proportion. A desired ratio of hydrocarbonaceous material to olefin-conditioning products can be achieved according to the invention, for example, by adjusting the flow rates of the hydrocarbonaceous material and the olefin-conditioning products, or by pre-mixing the appropriate amounts of hydrocarbonaceous material and olefin conditioning products.

In some embodiments, for example when solid hydrocarbonaceous materials are used, moisture 12 may optionally be removed from the feed composition prior to being fed to the reactor, e.g., by an optional dryer 14. Removal of moisture from the feed stream may be advantageous for several reasons. For example, the moisture in the feed stream may require additional energy input in order to heat the feed to a temperature sufficiently high to achieve pyrolysis. Variations in the moisture content of the feed may lead to difficulties in controlling the temperature of the reactor. In addition, removal of moisture from the feed can reduce or eliminate the need to process the water during later processing steps.

In some embodiments, the feed composition may be dried until the feed composition comprises less than about 10%, less than about 5%, less than about 2%, or less than about 1% water by weight. Suitable equipment capable of removing water from the feed composition is known to those skilled in the art. As an example, in one set of embodiments, the dryer comprises an oven heated to a particular temperature (e.g., at least about 80° C., at least about 100° C., at least about 150° C., or higher) through which the feed composition is continuously, semi-continuously, or periodically passed. In some cases, the dryer may comprise a vacuum chamber into which the feed composition is processed as a batch. Other embodiments of the dryer may combine elevated temperatures with vacuum operation. The dryer may be integrally connected to the reactor or may be provided as a separate unit from the reactor.

In some instances, the particle size of the feed composition may be reduced in an optional grinding system 16 prior to passing the feed to the reactor. In some embodiments, the average diameter of the ground feed composition exiting the grinding system may comprise no more than about 50%, not more than about 25%, no more than about 10%, no more than about 5%, no more than about 2% of the average diameter of the feed composition fed to the grinding system. Large-particle feed material may be more easily transportable and less difficult to process than small-particle feed material. On the other hand, in some cases it may be advantageous to feed small particles to the reactor (as discussed below). The use of a grinding system allows for the transport of large-particle feed between the source and the process, while enabling the feed of small particles to the reactor.

Suitable equipment capable of grinding the feed composition is known to those skilled in the art. For example, the grinding system may comprise an industrial mill (e.g., hammer mill, ball mill, etc.), a unit with blades (e.g., chipper, shredder, etc.), or any other suitable type of grinding system. In some embodiments, the grinding system may comprise a cooling system (e.g., an active cooling systems such as a pumped fluid heat exchanger, a passive cooling system such as one including fins, etc.), which may be used to maintain the feed composition at relatively low temperatures (e.g., ambient temperature) prior to introducing the feed composition to the reactor. The grinding system may be integrally connected to the reactor or may be provided as a separate unit from the reactor. While the grinding step is shown following the drying step in FIG. 2, the order of these operations may be reversed in some embodiments. In still other embodiments, the drying and grinding steps may be achieved using an integrated unit.

In some cases, grinding and cooling of the hydrocarbonaceous material may be achieved using separate units. Cooling of the hydrocarbonaceous material may be desirable, for example, to reduce or prevent unwanted decomposition of the feed material prior to passing it to the reactor. In one set of embodiments, the hydrocarbonaceous material may be passed to a grinding system to produce a ground hydrocarbonaceous material. The ground hydrocarbonaceous material may then be passed from the grinding system to a cooling system and cooled. The hydrocarbonaceous material may be cooled to a temperature of lower than about 300° C., lower than about 200° C., lower than about 100° C., lower than about 75° C., lower than about 50° C., lower than about 35° C., or lower than about 20° C. prior to introducing the hydrocarbonaceous material into the reactor. In embodiments that include the use of a cooling system, the cooling system includes an active cooling unit (e.g., a heat exchanger) capable of lowering the temperature of the biomass. In some embodiments, two or more of the drier, grinding system, and cooling system unit operations may be combined into a single unit. The cooling system may be, in some embodiments, directly integrated with one or more reactors.

As illustrated in FIG. 2, the feed composition may be transferred to reactor 20. The reactor may be used, in some instances, to perform catalytic pyrolysis of hydrocarbonaceous material. In the illustrative embodiment of FIG. 2, the reactor comprises any suitable reactor known to those skilled in the art. For example, in some instances, the reactor may comprise a continuously stirred tank reactor (CSTR), a batch reactor, a semi-batch reactor, or a fixed bed catalytic reactor, among others. In some cases, the reactor comprises a fluidized bed reactor, e.g., a circulating fluidized bed reactor, a moving bed reactor such as a riser reactor, or a bubbling bed reactor. Fluidized bed reactors may, in some cases, provide improved mixing of the catalyst and/or hydrocarbonaceous material during pyrolysis and/or subsequent reactions, which may lead to enhanced control over the reaction products formed. The use of fluidized bed reactors may also lead to improved heat transfer within the reactor. In addition, improved mixing in a fluidized bed reactor may lead to a reduction of the amount of coke adhered to the catalyst, resulting in reduced deactivation of the catalyst in some cases. Throughout this specification, various compositions are referred to as process streams; however, it should be understood that the processes could also be conducted in batch mode.

In some embodiments, at least a portion of the olefins in the fluid hydrocarbon product stream 30 is separated from the rest of the product stream to produce a recycle stream 100, comprising at least a portion of the separated olefins, and product stream 31A. The separation of olefins from fluid hydrocarbon products can be accomplished by an olefin recycler 102. While the olefin recycler is shown as being positioned directly downstream of reactor 20 in FIG. 2, it should be understood that the olefin recycler can be positioned at any point downstream of the reactor, and the separation of olefins from other fluid hydrocarbon products can potentially be performed at any of a variety of points after the fluid hydrocarbon products are produced. In addition, while recycle stream 100 is illustrated in FIG. 2 as being combined with feed stream 10 upstream of dryer 14, it should be understood that recycle stream 100 could alternatively be combined with feed stream 10 downstream of dryer 14 and/or grinder 16, fed directly to reactor 20, and/or combined with any of the catalyst streams (e.g., 34, 42, 44, 46, and/or 47) described in more detail below. Recycle stream 100 can be fed in part to the olefin conditioning step 80.

Suitable methods for separating olefins from other fluid hydrocarbon products are known to those of ordinary skill in the art. For example, olefins can be separated from other fluid hydrocarbon products by cooling product stream 30 to a temperature that lies between the boiling points of the olefins and the other fluid hydrocarbon products. Optionally, olefin recycler 102 can comprise a multi-stage separator. For example, the olefin recycler can comprise a first separator that directly separates the gaseous products (including olefins) from liquid products (e.g., high boiling point aromatics such as benzene, toluene, xylene, etc.), and a second separator that separates at least a portion of the olefins from other gaseous products (e.g., gaseous aromatics, CO2, CO, etc.). The methods and/or conditions used to perform the separation can depend upon the relative amounts and types of compounds present in the fluid hydrocarbon product stream, and one of ordinary skill in the art will be capable of selecting a method and the conditions suitable to achieve a given separation given the guidance provided herein.

In the set of embodiments illustrated in FIG. 2, separated catalyst may exit the solids separator via stream 34. In some cases, the catalyst exiting the separator may be at least partially deactivated. The separated catalyst may be fed, in some embodiments, to a regenerator 36 in which any catalyst that was at least partially deactivated may be re-activated. In some embodiments, the regenerator may comprise optional purge stream 37, which may be used to purge coke, ash, and/or catalyst from the regenerator. Methods for activating catalyst are well-known to those skilled in the art, for example, as described in Kirk-Othmer Encyclopedia of Chemical Technology (Online), Vol. 5, Hoboken, N.J.: Wiley-Interscience, 2001, pages 255-322.

In one set of embodiments, an oxidizing agent is fed to the regenerator via a stream 38, e.g., as shown in FIG. 2. The oxidizing agent may originate from any source including, for example, a tank of oxygen, atmospheric air, or steam, among others. In the regenerator, the catalyst is re-activated by reacting the catalyst with the oxidizing agent. In some cases, the deactivated catalyst may comprise residual carbon and/or coke, which may be removed via reaction with the oxidizing agent in the regenerator. The regenerator in FIG. 2 comprises a vent stream 40 which may include regeneration reaction products, residual oxidizing agent, etc. The vent stream from the regenerator may be passed through a catalytic exhaust gas cleanup system to further reduce the concentrations of CO and hydrocarbons to reduce emissions vented to the atmosphere. Portions of the vent stream 40 may be recycled to the gas feed of the regenerator 38 to control the heat release of the regeneration process.

The regenerator may be of any suitable size mentioned above in connection with the reactor or the solids separator. In addition, the regenerator may be operated at elevated temperatures in some cases (e.g., at least about 300° C., 400° C., 500° C., 600° C., 700° C., 800° C., or higher). The residence time of the catalyst in the regenerator may also be controlled using methods known by those skilled in the art, including those outlined above. In some instances, the mass flow rate of the catalyst through the regenerator will be coupled to the flow rate(s) in the reactor and/or solids separator in order to preserve the mass balance in the system.

As shown in the illustrative embodiment of FIG. 2, the regenerated catalyst may exit the regenerator via stream 42. The regenerated catalyst may be recycled back to the reactor via recycle stream 47. In some cases, catalyst may be lost from the system during operation. In some such and other cases, additional “makeup” catalyst may be added to the system via a makeup stream 46. As shown illustratively in FIG. 2, the regenerated and makeup catalyst may be fed to the reactor with the fluidization fluid via recycle stream 47, although in other embodiments, the catalyst and fluidization fluid may be fed to the reactor via separate streams.

Referring back to solids separator 32 in FIG. 2, the reaction products (e.g., fluid hydrocarbon products) exit the solids separator via stream 48. In some cases, a fraction of stream 48 may be purged via purge stream 60. The contents of the purge stream may be fed to a combustor or a water-gas shift reactor, for example, to recuperate energy that would otherwise be lost from the system. In some cases, the reaction products in stream 48 may be fed to an optional condenser 50. The condenser may comprise a heat exchanger which condenses at least a portion of the reaction products from a gaseous to a liquid state. The condenser may be used to separate the reaction products into gaseous, liquid, and solid fractions. The operation of condensers is well known to those skilled in the art. Examples of condensers are described in more detail in Perry's Chemical Engineers' Handbook, Section 11: “Heat Transfer Equipment.” 8th ed. New York: McGraw-Hill, 2008.

The condenser may also, in some embodiments, make use of pressure change to condense portions of the product stream. In FIG. 2, stream 54 may comprise the liquid fraction of the reaction products (e.g., water, aromatic compounds, olefin compounds, etc.), and stream 74 may comprise the gaseous fraction of the reaction products (e.g., CO, CO2, H2, ethylene, propylene, butenes, etc.). In some embodiments, the gaseous fraction may be fed to a vapor recovery system 70. The vapor recovery system may be used, for example, to recover any selected vapors within stream 74 and transport them via stream 72. In addition, stream 76 may be used to transport CO, CO2, and/or other non-recoverable gases from the vapor recovery system. It should be noted that, in some embodiments, the optional vapor recovery system may be placed in other locations. For example, in some embodiments, a vapor recovery system may be positioned downstream of purge stream 54.

Other products (e.g., excess gas) may be transported to optional compressor 26 via stream 56, where they may be compressed and used as fluidization gas in the reactor (stream 22) and/or where they may assist in transporting the hydrocarbonaceous material to the reactor (stream 58). In some instances, the liquid fraction may be further processed, for example, to separate the water phase from the organic phase, to separate individual compounds, etc.

It should be understood that, while the set of embodiments described by FIG. 2 includes a reactor, solids separator, regenerator, condenser, etc., not all embodiments will involve the use of these elements. For example, in some embodiments, the feed stream may be fed to a catalytic fixed bed reactor, reacted, and the reaction products may be collected directly from the reactor and cooled without the use of a dedicated condenser. In some instances the product may be fed to a quench tower to which is fed a cooling fluid, preferably a liquid, along with the product stream to cool and condense the products. In some instances, while a dryer, grinding system, solids separator, regenerator, condenser, and/or compressor may be used as part of the process, one or more of these elements may comprise separate units not fluidically and/or integrally connected to the reactor. In other embodiments, one or more of the dryer, grinding system, solids separator, regenerator, condenser, and/or compressor may be absent. In some embodiments, the desired reaction product(s) (e.g., liquid aromatic hydrocarbons, olefin hydrocarbons, gaseous products, etc.) may be recovered at any point in the production process (e.g., after passage through the reactor, after separation, after condensation, etc.).

### BRIEF DESCRIPTION OF THE DRAWINGS

FIG. 1 schematically illustrates a prior art system by Huber et al. for fast catalytic pyrolysis.

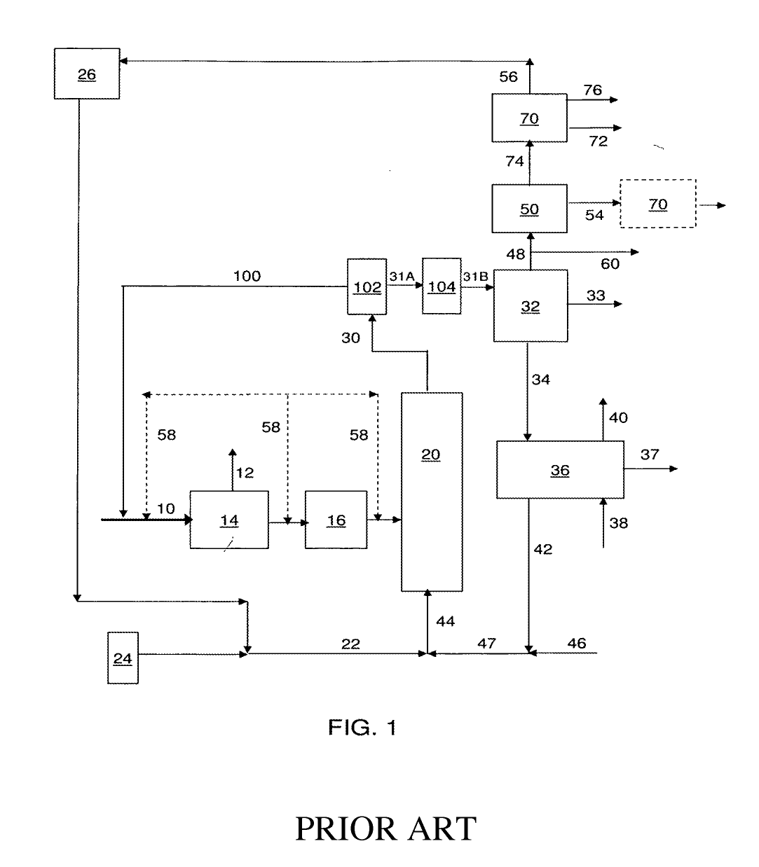

FIG. 2 schematically illustrates a system of the present invention for pyrolysis of biomass that includes an olefin-conditioning step.

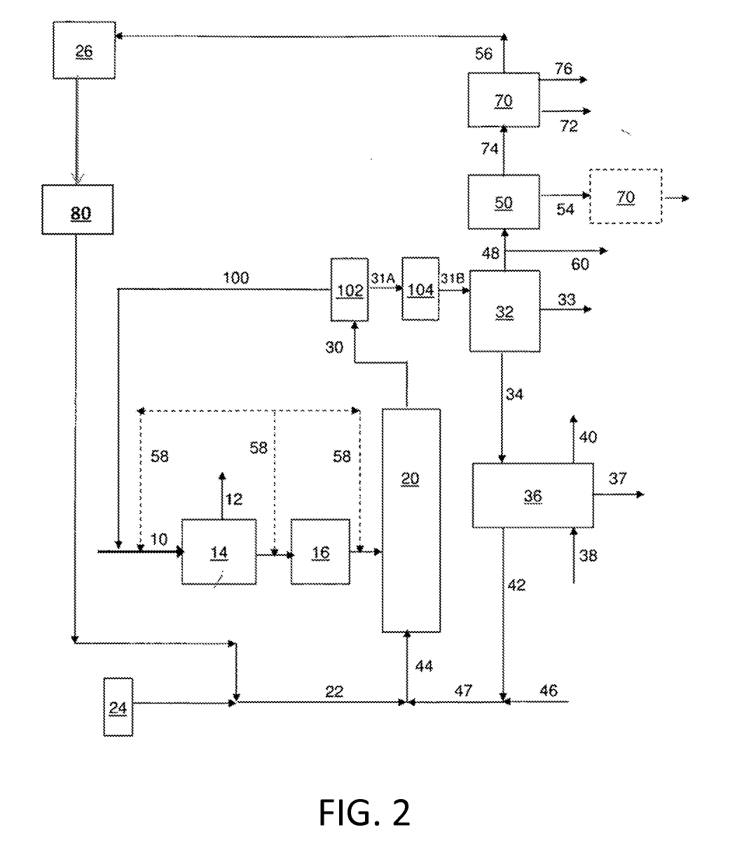

### GLOSSARY

### Aromatics

As used herein, the terms “aromatics” or “aromatic compound” are used to refer to a hydrocarbon compound or compounds comprising one or more aromatic groups such as, for example, single aromatic ring systems (e.g., benzyl, phenyl, etc.) and fused polycyclic aromatic ring systems (e.g. naphthyl, 1,2,3,4-tetrahydronaphthyl, etc.). Examples of aromatic compounds include, but are not limited to, benzene, toluene, indane, indene, 2-ethyl toluene, 3-ethyl toluene, 4-ethyl toluene, trimethyl benzene (e.g., 1,3,5-trimethyl benzene, 1,2,4-trimethyl benzene, 1,2,3-trimethyl benzene, etc.), ethylbenzene, styrene, cumene, methylbenzene, propylbenzene, xylenes (e.g., p-xylene, m-xylene, o-xylene, etc.), naphthalene, methyl-naphthalene (e.g., 1-methyl naphthalene, anthracene, 9.10-dimethylanthracene, pyrene, phenanthrene, dimethyl-naphthalene (e.g., 1,5-dimethylnaphthalene, 1,6-dimethylnaphthalene, 2,5-dimethylnaphthalene, etc.), ethyl-naphthalene, hydrindene, methyl-hydrindene, and dymethyl-hydrindene. Single-ring and/or higher ring aromatics may also be produced in some embodiments.

### Biomass

As used herein, the term “biomass” is given its conventional meaning in the art and is used to refer to any organic source of energy or chemicals that is renewable. Its 10 major components can be: (1) trees (wood) and all other vegetation; (2) agricultural products and wastes (corn, fruit, garbage ensilage, etc.); (3) algae and other marine plants; (4) metabolic wastes (manure, sewage), and (5) cellulosic urban waste. Examples of biomass materials are described, for example, in Huber, G. W. et al, “Synthesis of Transportation Fuels from Biomass: Chemistry, Catalysts, and Engineering,” Chem. Rev. 106, (2006), pp. 4044-4098.

Biomass is conventionally defined as the living and recently dead biological material that can be converted for use as fuel or for industrial production. The criterion as biomass is that the material should be recently participating in the carbon cycle so that the release of carbon in the combustion process results in no net increase averaged over a reasonably short period of time (for this reason, fossil fuels such as peat, lignite and coal are not considered biomass by this definition as they contain carbon that has not participated in the carbon cycle for a long time so that their combustion results in a net increase in atmospheric carbon dioxide). Most commonly, biomass refers to plant matter grown for use as biofuel, but it also includes plant or animal matter used for production of fibers, chemicals or heat. Biomass may also include biodegradable wastes or byproducts that can be burnt as fuel or converted to chemicals, including municipal wastes, green waste (the biodegradable waste comprised of garden or park waste, such as grass or flower cuttings and hedge trimmings), byproducts of farming including animal manures, food processing wastes, sewage sludge, black liquor from wood pulp or algae. Biomass excludes organic material which has been transformed by geological processes into substances such as coal, oil shale or petroleum. Biomass is widely and typically grown from plants, including miscanthus, spurge, sunflower, switchgrass, hemp, corn (maize), poplar, willow, sugarcane, and oil palm (palm oil) with the roots, stems, leaves, seed husks and fruits all being potentially useful. The particular plant or other biomass source used is not important to the product chemical or fuel although the processing of the raw material for introduction to the processing unit will vary according to the needs of the unit and the form of the biomass.

### Biomass Feed Particle Sizes

The hydrocarbonaceous material in the feed composition may comprise a solid, liquid, and/or gas. In cases where the hydrocarbonaceous material includes solids, the solids may be of any suitable size. In some cases, it may be advantageous to use hydrocarbonaceous solids with relatively small particle sizes. Small-particle solids may, in some instances, react more quickly than larger solids due to their relatively higher surface area-to-volume ratios compared to larger solids. In addition, small particle sizes may allow for more efficient heat transfer within each particle and/or within the reactor volume. This may prevent or reduce the formation of undesired reaction products. Moreover, small particle sizes may provide for increased solid-gas and solid-solid contact, leading to improved heat and mass transfer.

### Biomass Pyrolysis Liquid

Biomass pyrolysis liquid or bio-oil is the liquid fraction that can be isolated from a pyrolysis reaction of biomass. Biomass pyrolysis liquid is usually dark brown and approximates to biomass in elemental composition. It is composed of a very complex mixture of oxygenated hydrocarbons with an appreciable proportion of water from both the original moisture and reaction product. Compositionally, the biomass pyrolysis oil will vary with the type of biomass, but is known to consist of oxygenated low molecular weight alcohols (e.g., furfuryl alcohol), aldehydes (aromatic aldehydes), ketones (furanone), phenols (methoxy phenols) and water. Solid char may also be present, suspended in the oil. The liquid is formed by rapidly quenching the intermediate products of flash pyrolysis of hemicellulose, cellulose and lignin in the biomass. Chemically, the oil contains several hundred different chemicals in widely varying proportions, ranging from formaldehyde and acetic acid to complex, high molecular weight phenols, anhydrosugars and other oligosaccharides. It has a distinctive odor from low molecular weight aldehydes and acids, is usually acidic with a pH of 1.5-3.8, and can be an irritant.

### Catalyst Residence Time

The catalyst residence time of the catalyst in the reactor is defined as the volume of the reactor filled with catalyst divided by the volumetric flow rate of the catalyst through the reactor. For example, if a 3-liter reactor contains 2 liters of catalyst and a flow of 0.4 liters per minute of catalyst is fed through the reactor, i.e., both fed and removed, the catalyst residence time is 2/0.4 minutes, or 5 minutes.

### Contact Time

Contact time is the residence time of a material in a reactor or other device, when measured or calculated under standard conditions of temperature and pressure, i.e., 0° C. and 1 atm. For example, a 2-liter reactor to which is fed 3 standard liters per minute of gas has a contact time of 2/3 minute, or 40 seconds for that gas. For a chemical reaction, contact time or residence time is based on the volume of the reactor, where substantial reaction is occurring, and would exclude volume where substantially no reaction is occurring, such as an inlet or an exhaust conduit. For catalyzed reactions, the volume of a reaction chamber is the volume where catalyst is present.

### Conversion

The term “conversion of a reactant” refers to the reactant mole or mass change between a material flowing into a reactor and a material flowing out of the reactor divided by the moles or mass of reactant in the material flowing into the reactor. For example, if 100 grams of ethylene are fed to a reactor and 30 grams of ethylene are flowing out of the reactor, the conversion is [(100−30)/100]=70% conversion of ethylene.

### Fluid

The term “fluid” refers to a gas, a liquid, a mixture of a gas and a liquid, or a gas or a liquid containing dispersed solids, liquid droplets and/or gaseous bubbles. The terms “gas” and “vapor” have the same meaning and are sometimes used interchangeably. In some embodiments, it may be advantageous to control the residence time of the fluidization fluid in the reactor. The fluidization residence time of the fluidization fluid is defined as the volume of the reactor divided by the volumetric flow rate of the fluidization fluid under process conditions of temperature and pressure.

### Fluidized Bed Reactor

As used herein, the term “fluidized bed reactor” is given its conventional meaning in the art and is used to refer to reactors comprising a vessel that can contain a granular solid material (e.g., silica particles, catalyst particles, etc.), in which a fluid (e.g., a gas or a liquid) is passed through the granular solid material at velocities sufficiently high as to suspend the solid material and cause it to behave as though it were a fluid. Examples of fluidized bed reactors are described in Kirk-Othmer Encyclopedia of Chemical Technology (online), Vol. 11, Hoboken, N.J.: Wiley-Interscience, 2001, pages 791-825, incorporated herein by reference. The term “circulating fluidized bed reactor” is also given its conventional meaning in the art and is used to refer to fluidized bed reactors in which the granular solid material is passed out of the reactor, circulated through a line in fluid communication with the reactor, and recycled back into the reactor. Examples of circulating fluidized bed reactors are described in Kirk-Othmer Encyclopedia of Chemical Technology (Online), Vol. 11, Hoboken, N.J.: Wiley-Interscience, 2001, pages 791-825.

Bubbling fluidized bed reactors and turbulent fluidized bed reactors are also known to those skilled in the art. In bubbling fluidized bed reactors, the fluid stream used to fluidize the granular solid material is operated at a sufficiently low flow rate such that bubbles and voids are observed within the volume of the fluidized bed during operation. In turbulent fluidized bed reactors, the flow rate of the fluidizing stream is higher than that employed in a bubbling fluidized bed reactor, and hence, bubbles and voids are not observed within the volume of the fluidized bed during operation.

Examples of bubbling and turbulent fluidized bed reactors are described in Kirk-Othmer Encyclopedia of Chemical Technology (online), Vol. 11, Hoboken, N.J.: Wiley [symbol] Interscience, c2001-, pages 791-825, incorporated herein by reference.

### Olefins

As used herein, the terms “olefin” or “olefin compound” (a.k.a. “alkenes”) are given their ordinary meaning in the art, and are used to refer to any unsaturated hydrocarbon containing one or more pairs of carbon atoms linked by a double bond. Olefins include both cyclic and acyclic (aliphatic) olefins, in which the double bond is located between carbon atoms forming part of a cyclic (closed-ring) or of an open-chain grouping, respectively. In addition, olefins may include any suitable number of double bonds (e.g., monoolefins, diolefins, triolefins, etc.). Examples of olefin compounds include, but are not limited to, ethene, propene, allene (propadiene), 1-butene, 2-butene, isobutene (2 methyl propene), butadiene, and isoprene, among others. Examples of cyclic olefins include cyclopentene, cyclohexane, cycloheptene, among others. Aromatic compounds such as toluene are not considered olefins; however, olefins that include aromatic moieties are considered olefins, for example, benzyl acrylate or styrene.

### Pore Size

Pore size relates to the size of a molecule or atom that can penetrate into the pores of a material. As used herein, the term “pore size” for zeolites and similar catalyst compositions refers to the Norman radii adjusted pore size well known to those skilled in the art. Determination of Norman radii adjusted pore size is described, for example, in Cook, M.; Conner, W. C., “How big are the pores of zeolites?” Proceedings of the International Zeolite Conference, 12th, Baltimore, Jul. 5-10, 1998; (1999), 1, pp 409-414, which is incorporated herein by reference in its entirety. As a specific exemplary calculation, the atomic radii for ZSM-5 pores are about 5.5-5.6 Angstroms, as measured by x-ray diffraction. In order to adjust for the repulsive effects between the oxygen atoms in the catalyst, Cook and Conner have shown that the Norman adjusted radii are 0.7 Angstroms larger than the atomic radii (about 6.2-6.3 Angstroms).

One of ordinary skill in the art will understand how to determine the pore size (e.g., minimum pore size, average of minimum pore sizes) in a catalyst. For example, x-ray diffraction (XRD) can be used to determine atomic coordinates. XRD techniques for the determination of pore size are described, for example, in Pecharsky, V. K. et at, “Fundamentals of Powder Diffraction and Structural Characterization of Materials,” Springer Science+Business Media, Inc., New York, 2005, incorporated herein by reference in its entirety. Other techniques that may be useful in determining pore sizes (e.g., zeolite pore sizes) include, for example, helium pycnometry or low-pressure argon adsorption techniques. These and other techniques are described in Magee, J. S. et at, “Fluid Catalytic Cracking: Science and Technology,” Elsevier Publishing Company, Jul. 1, 1993, pp. 185-195, which is incorporated herein by reference in its entirety. Pore sizes of mesoporous catalysts may be determined using, for example, nitrogen adsorption techniques, as described in Gregg, S. J. at al, “Adsorption, Surface Area and Porosity,” 2nd Ed., Academic Press Inc., New York, 1982 and Rouquerol, F. et al, “Adsorption by powders and porous materials. Principles, Methodology and Applications,” Academic Press Inc., New York, 1998, both incorporated herein by reference in their entirety.

In some embodiments, a screening method is used to select catalysts with appropriate pore sizes for the conversion of specific pyrolysis product molecules. The screening method may comprise determining the size of pyrolysis product molecules desired to be catalytically reacted (e.g., the molecule kinetic diameters of the pyrolysis product molecules). One of ordinary skill in the art can calculate, for example, the kinetic diameter of a given molecule. The type of catalyst may then be chosen such that the pores of the catalyst (e.g., Norman adjusted minimum radii) are sufficiently large to allow the pyrolysis product molecules to diffuse into and/or react with the catalyst. In some embodiments, the catalysts are chosen such that their pore sizes are sufficiently small to prevent entry and/or reaction of pyrolysis products whose reaction would be undesirable.

### Catalysts

Catalyst components useful in the context of this invention can be selected from any catalyst known in the art, or as would be understood by those skilled in the art. Catalysts promote and/or effect reactions. Thus, as used herein, catalysts lower the activation energy (increase the rate) of a chemical process, and/or improve the distribution of products or intermediates in a chemical reaction (for example, a shape selective catalyst). Examples of reactions that can be catalyzed include: dehydration, dehydrogenation, isomerization, hydrogen transfer, aromatization, decarbonylation, decarboxylation, aldol condensation, and combinations thereof. Catalyst components can be considered acidic, neutral or basic, as would be understood by those skilled in the art.

For fast catalytic pyrolysis, particularly advantageous catalysts include those containing internal porosity selected according to pore size (e.g., mesoporous and pore sizes typically associated with zeolites), e.g., average pore sizes of less than about 100 Angstroms, less than about 50 Angstroms, less than about 20 Angstroms, less than about 10 Angstroms, less than about 5 Angstroms, or smaller. In some embodiments, catalysts with average pore sizes of from about 5 Angstroms to about 100 Angstroms may be used. In some embodiments, catalysts with average pore sizes of between about 5.5 Angstroms and about 6.5 Angstroms, or between about 5.9 Angstroms and about 6.3 Angstroms may be used. In some cases, catalysts with average pore sizes of between about 7 Angstroms and about 8 Angstroms, or between about 7.2 Angstroms and about 7.8 Angstroms may be used.

In some preferred embodiments of FCP, the catalyst may be selected from naturally occurring zeolites, synthetic zeolites and combinations thereof. In certain embodiments, the catalyst may be a ZSM-5 zeolite catalyst, as would be understood by those skilled in the art. Optionally, such a catalyst can comprise acidic sites. Other types of zeolite catalysts include: ferrierite, zeolite Y, zeolite beta, mordenite, MCM-22, ZSM-23, ZSM-57, SUZ-4, EU-1, ZSM-11, (S)AlP0-31, SSZ-23, among others. In other embodiments, non-zeolite catalysts may be used; for example, WOx/ZrO2, aluminum phosphates, etc. In some embodiments, the catalyst may comprise a metal and/or a metal oxide. Suitable metals and/or oxides include, for example, nickel, palladium, platinum, titanium, vanadium, chromium, manganese, iron, cobalt, zinc, copper, gallium, and/or any of their oxides, among others. In some cases promoter elements chosen from among the rare earth elements, i.e., elements 57-71, cerium, zirconium or their oxides for combinations of these may be included to modify activity or structure of the catalyst. In addition, in some cases, properties of the catalysts (e.g., pore structure, type and/or number of acid sites, etc.) may be chosen to selectively produce a desired product.

Catalysts for other processes, such as alkylation of olefins are well-known and can be selected for the treatment processes described herein.

### Pyrolysis

As used herein, the terms “pyrolysis” and “pyrolyzing” are given their conventional meaning in the art and are used to refer to the transformation of a compound, e.g., a solid hydrocarbonaceous material, into one or more other substances, e.g., volatile organic compounds, gases and coke, by heat, preferably without the addition of, or in the absence of, O₂. Preferably, the volume fraction of O2 present in a pyrolysis reaction chamber is 0.5% or less. Pyrolysis may take place with or without the use of a catalyst. “Catalytic pyrolysis” refers to pyrolysis performed in the presence of a catalyst, and may involve steps as described in more detail below. Example of catalytic pyrolysis processes are outlined, for example, in Huber, G. W. et al, “Synthesis of Transportation Fuels from Biomass: Chemistry, Catalysts, and Engineering,” Chem. Rev. 106, (2006), pp. 4044-4098.

### Residence Time.

In some instances, it is beneficial to control the residence time of the reactants (e.g., the solid hydrocarbonaceous material and/or a non-solid reactant) and catalyst(s) in a reactor and/or under a defined set of reaction conditions (i.e. conditions under which the reactants can undergo pyrolysis or catalysis in a given reactor system).

An overall residence time is defined as the volume of the reactor or device, or specific portion of a device, divided by the exit flow of all gases out of the reactor, or device including fluidization gas, products, and impurities, measured or calculated at the average temperature of the reactor or device and the exit pressure of the reactor or device.

A reactant residence time of a reactant in the reactor is defined as the amount of time the reactant spends in the reactor. As is conventional, residence time is based on the feed rate of reactant and is independent of rate of reaction. The reactant residence time of the reactants in a reactor may be calculated using different methods depending upon the type of reactor being used. For gaseous reactants, where flow rate into the reactor is known, this is typically a simple calculation. In the case of solid reactants in which the reactor comprises a packed bed reactor into which only reactants are continuously fed (i.e. no carrier or fluidizing flow is utilized), the reactant residence time in the reactor may be calculated by dividing the volume of the reactor by the volumetric flow rate of the hydrocarbonaceous material and fluid hydrocarbon product exiting the reactor.

In cases where the reaction takes place in a reactor that is closed to the flow of mass during operation (e.g., a batch reactor), the batch residence time of the reactants in such a reactor is defined as the amount of time elapsing between the time at which the temperature in the reactor containing the reactants reaches a level sufficient to commence a pyrolysis reaction (e.g., for FCP, typically about 300° C. to about 1000° C. for many typical hydrocarbonaceous feedstock materials) and the time at which the reactor is quenched (e.g., cooled to a temperature below that sufficient to support further pyrolysis—e.g. typically about 300° C. to about 1000° C. for many typical hydrocarbonaceous feedstock materials).

In some cases, e.g. for certain fluidized bed reactors, the reactor feed stream(s) may include feed stream(s) comprising auxiliary materials (i.e., matter other than solid hydrocarbonaceous materials and/or non-solid reactants). For example, in certain cases where fluidized beds are used as reactors, the feed stream may comprise fluidization fluid(s). In cases where circulating fluidized beds are used, catalyst and the fluidization fluid may both be fed, recycled, or fed and recycled to the reactor. In such cases, the reactant residence time of the reactants in the reactor can be determined as the volume of the reactor divided by the volumetric flow rate of the reactants and reaction product gases exiting the reactor as with the packed bed situation described above; however, since the flow rates of the reactants and reaction product gases exiting the reactor may not be convenient to determine directly, the volumetric flow rate of the reactants and reaction product gases exiting the reactor may be estimated by subtracting the feed volumetric flow rate of the auxiliary materials into the reactor (e.g., fluidization fluid, catalyst, contaminants, etc.) into the reactor from the total volumetric flow rate of the gas stream(s) exiting the reactor.

### Selectivity

The term “selectivity” refers to the amount of production of a particular product in comparison to a selection of products. Selectivity to a product may be calculated by dividing the amount of the particular product by the amount of a number of products produced. For example, if 75 grams of aromatics are produced in a reaction and 20 grams of benzene are found in these aromatics, the selectivity to benzene amongst aromatic products is 20/75=26.7%. Selectivity can be calculated on a mass basis, as in the aforementioned example, or it can be calculated on a carbon basis, where the selectivity is calculated by dividing the amount of carbon that is found in a particular product by the amount of carbon that is found in a selection of products. Unless specified otherwise, for reactions involving biomass as a reactant, selectivity is on a mass basis. For reactions involving conversion of a specific molecular reactant (ethene, for example), selectivity is the percentage (on a mass basis unless specified otherwise) of a selected product divided by all the products produced.

### Yield

The term yield is used herein to refer to the amount of a product flowing out of a reactor divided by the amount of reactant flowing into the reactor, usually expressed as a percentage or fraction. Yields are often calculated on a mass basis, carbon basis, or on the basis of a particular feed component. Mass yield is the mass of a particular product divided by the weight of feed used to prepare that product. For example, if 500 grams of biomass is fed to a reactor and 45 grams of benzene is produced, the mass yield of benzene would be 45/500=9% benzene. Carbon yield is the mass of carbon found in a particular product divided by the mass of carbon in the feed to the reactor. For example, if 500 grams of biomass that contains 40% carbon is reacted to produce 45 grams of benzene that contains 92.3% carbon, the carbon yield is [(45*0.923)/(500*0.40)]=20.8%. Carbon yield from biomass is the mass of carbon found in a particular product divided by the mass of carbon fed to the reactor in a particular feed component. For example, if 500 grams of biomass containing 40% carbon and 100 grams of CO2 are reacted to produce 40 g of benzene (containing 92.3% carbon), the carbon yield on biomass is [(40*0.923)/(500*0.40)]=18.5%; note that the mass of CO₂ does not enter into the calculation.

As is standard patent terminology, the term “comprising” means “including” and does not exclude additional components. Any of the inventive aspects described in conjunction with the term “comprising” also include narrower embodiments in which the term “comprising” is replaced by the narrower terms “consisting essentially of” or “consisting of:” As used in this specification, the terms “includes” or “including” should not be read as limiting the invention but, rather, listing exemplary components.

### DESCRIPTION OF SOME PREFERRED EMBODIMENTS

Since olefins are commonly produced, the invention is generally applicable to any biomass pyrolysis reaction. Preferably, the biomass feedstock comprises a solid hydrocarbonaceous material. The biomass feedstock may comprise, for example, any one or combination of the biomass sources that are mentioned in the Glossary section. The pyrolysis reactor can be without a solid catalyst; however, preferably, the pyrolysis reactor comprises a solid catalyst for fast catalytic pyrolysis (FCP). The type of reactor and the type of solid catalyst (if present) are not limited, and can be generally of the type known for conversion of biomass to fluid hydrocarbonaceous streams. Examples of suitable apparatus and process conditions for FCP are described in the patent application of Huber at al. [US20090227823] that is incorporated herein by reference. Conditions for FCP of biomass can be selected from any one or any combination of the following features (which are not intended to limit the broader aspects of the invention): a zeolite catalyst, a ZSM-5 catalyst; a zeolite catalyst comprising one or more of the following metals: titanium, vanadium, chromium, manganese, iron, cobalt, nickel, copper, zinc, gallium, platinum, palladium, silver, phosphorus, sodium, potassium, magnesium, calcium, tungsten, zirconium, cerium, lanthanum, and combinations thereof; a fluidized bed, circulating bed, or riser reactor; an operating temperature in the range of 300° to 1000° C.; and/or a solid catalyst-to-biomass mass ratio of between 0.1 and 20.

The olefin-containing fraction (i.e., the “first fraction”) can have a wide variety of compositions; provided that this fraction contains a significant amount of olefins. The fraction could simply be the gaseous (noncondensed) fraction that includes CO, CO2, ethylene, propylene and numerous other components and may include higher olefins. The olefin-containing fraction could also contain alkynes such as ethyne, propyne, butyne or the like. In other embodiments, the fraction could be a relatively olefin-rich stream that is separated from a relatively olefin-poor stream. Examples of separation techniques that can be used in a biomass conversion system include: cryogenic separation, distillation, membrane separation, adsorptive separation or reactive separation. The olefin-containing fraction may also arise from further processing of solids or liquids that are obtained downstream of the pyrolytic reactor. In some preferred embodiments, the olefin-containing stream comprises at least 2 mass % olefins, in some embodiments, at least 5 mass % olefins, and in some embodiments, in the range of 2 to 20 mass % olefins or more.

Other gases in the olefin-containing fraction could include methane, ethane, propane, CO, CO2, water, propadiene, methyl acetylene, H₂ and/or N₂.

In some embodiments, treatment of the olefin-containing stream occurs in a catalyst-containing reactor such as a packed bed reactor. Treatment reactions can include (but are not limited to) one or more of the following reactions: hydrogenation, hydrolysis, hydroformylation, cyclization, dimerization, and/or polymerization. The treatment process can be conducted at lower temperatures than the FCP process in a secondary reactor or in a zone of the FCP reactor that is at lower temperatures; temperatures during treatment are preferably maintained below 400° C., and are preferably in the range 80-400° C.

In some embodiments, the olefin-containing stream is contacted with an acid catalyst at conditions to cause dimerization or oligomerization. Acid catalysts can be chosen from among those known to those skilled in the art, including liquid acids like: H2SO4 or HNO3, supported acids such as sulfated zirconia, or solid acids such as solid phosphoric acid, zeolites, pillared clays, or amorphous silica-alumina mixtures. Preferred catalysts comprise solid phosphoric acid (such as phosphoric acid on kieselguhr) or zeolites ZSM5, ZSM11, ZSM12, ZSM22, ZSM23, ZSM35, ZSM49, and MCM56. Regenerated catalyst can be used, including regenerated ZSM5 from the FCP process. A preferred temperature range is 120 to 300° C., more preferably 150 to 250° C.; although higher temperatures could be used. Pressures preferably are in the range of 1 atm to 20 atm, more preferably 1-5 atm a. Higher pressures can be used if high conversion of the olefins is desired. Space velocity for the dimerization/oligomerization is preferably in the range of 5 to 30 GHSV. The reaction can be conducted in various types of reactors, but preferably is conducted in a fixed-bed reactor. This reaction is most effective for the conversion of C3 and higher; thus for a mixture comprising ethylene and propylene, typically more of the propylene is consumed.

In some embodiments the olefin-containing stream is contacted with catalyst that has been removed from the FCP reactor prior to the catalyst being regenerated. In this case the olefin-containing stream is contacted with the used catalyst that is partially deactivated or coked, after it has been cooled below typical FCP process conditions. A preferred temperature range is 120 to 300° C., more preferably 150 to 250° C.; although higher temperatures could be used. Pressures preferably are in the range of 1 atm to 20 atm, more preferably 1-5 atm. After it has contacted the used catalyst the olefin-containing stream can be fed to the FCP reactor or it can be used as the fluidization fluid for the FCP reactor. An additional advantage of contacting the olefin-containing stream with the used catalyst before regeneration is that aromatic compounds or other useful products that are adsorbed on the catalyst may be recovered in the olefin-containing stream and returned to the reactor. The conditioned olefin-containing stream may be pre-heated prior to being fed to the FCP reactor.

In some embodiments, the product stream is hydrogenated and the liquid fuel fraction collected before recycle of the gaseous components; this may be particularly advantageous if catalyst is at least partially regenerated in the hydrogenation prior to recycling to a FCP reactor.

In some embodiments, ethylene may be converted to propylene prior to recycle to a FCP reactor. One example of such a conversion is described by Coperet et al., in U.S. Pat. No. 7,638,672 which is incorporated herein by reference as if reproduced in full below.

In another alternative, the olefins are treated by alkylation. In this case, the olefin-containing stream is preferably mixed with an aromatic-containing stream and contacted with an acid catalyst. Acid catalysts can be chosen from among those known to those skilled in the art including liquid acids like H2SO4 or HNO3, supported acids, such as sulfated zirconia, or solid acids such as solid phosphoric acid, zeolites, pillared clays, or amorphous silica-alumina mixtures. Preferred catalysts include: zeolite Y, such as ultrastable zeolite Y (USY), or zeolites ZSM5, ZSM11, ZSM12, ZSM22, ZSM23, ZSM35, and ZSM49. The zeolite catalyst is typically present along with a binder such as silica, alumina, or zirconia, or mixtures thereof. The catalysts can be promoted with metals to improve performance and limit coke deposition. Any metal from those of atomic numbers 21-31, 57-71 and the precious metals Pd, Pt, Ag or Au can be used, or combinations thereof, more preferably the catalyst comprises Pd, Pt, or combinations thereof. Combinations of Pt and/or Pd with Ru, Ir, Rh, Cu, Re, Ag and/or Au are also desirable. More preferably, in addition to Pt and/or Pd, the catalyst comprises Ni, Co, Mn, Cr, V, Fe, Ti, or combinations thereof. Preferably, the catalyst contains 0.1 to 2 mass percent of the above-mentioned metals. Regenerated catalyst can be used, including regenerated ZSM5 or other catalyst from the FCP process. A preferred temperature range is 120 to 300° C., more preferably 150 to 250° C.; although higher temperatures could be used. Pressures preferably are in the range from 1 atm to 20 atm, with pressures of 1-5 atm more preferred. Higher pressures can be used if high conversion of the olefins is desired. In some preferred embodiments, the olefin-containing stream is contacted with a liquid, aromatic-containing stream that extracts olefins from the olefin-containing stream; then the alkylation of olefins in the aromatic-containing stream is conducted simultaneously with or, more preferably, subsequent to the extraction step. Preferably, at least a portion, and more preferably all of the aromatic compounds are derived from the product stream of an FCP reactor. The molar ratio of aromatic compounds to olefins is preferably greater than 1, more preferably greater than 5. The alkylation reaction may be conducted in a variety of reactor types, preferably a fixed-bed reactor, and in some embodiments, the olefins are fed at multiple points along the length of a fixed-bed reactor.

In some embodiments, the olefin-containing stream is subjected to both dimerization/oligomerization and alkylation. These processes could be conducted in series (in either order). Alternatively, the olefin-containing stream could be split and separate fractions treated with dimerization/oligomerization and alkylation and the resulting product streams could be recombined prior to (or, less preferably) simultaneous with recycle into a FCP reactor.

In some embodiments, a radical inhibitor is added to the olefin-containing stream or added to the treated gas before it is admitted to the pyrolysis reactor (typically a FCP reactor) so that the olefins do not polymerize in the reactor before contacting the FCP catalyst(s) in the FCP reactor. Radical inhibitors include alcohol, acid (especially acetic acid), ether, ester, hydroquinone, substituted hydroquinone, nitrobenzene, diethylhydroxylamine (DEHA), di(phenyl)-(2,4,6-trinitrophenyl)iminoazanium (DPPH), butylhydroxytoluene (BHT), 4-hydroxy-2,2,6,6-tetramethylpiperidin-1-oxyl (4-hydroxy-TEMPO), or other radical inhibiting agent before it is recycled to a reactor. Phenol or any substituted phenol, preferably a phenol or substituted phenol present in or derived from the FCP reaction products, can be used as a radical inhibitor. The radical inhibitor can be passed into the FCP reactor where it may undergo further upgrading to produce additional aromatic products. Inclusion of a radical inhibitor can permit the olefin-containing stream to be heated to higher temperatures before recycle to the FCP reactor than can be used in the absence of the inhibitor. The recycle stream can be heated to temperatures above 150° C., or above 300° C., or even to temperatures above 400° C. before entering the FCP reactor. The invention also includes the use of radical inhibitors in the olefin stream—generally for any process occurring subsequent to biomass FCP that may suffer from olefin polymerization; although the use of radical inhibitors, that are obtained from the FCP products, are especially advantageous for recycle to the FCP reactor since some these compounds can serve the dual purpose of inhibiting polymerization and upgrading to a fuel.

In another embodiment the fraction containing olefins is mixed with water vapor and passed over a hydrolysis catalyst effective for the hydrolysis of olefins at a lower temperature before being fed to a pyrolysis reactor, preferably recycled to the same FCP reactor. The hydrolysis catalyst is a catalyst that catalyzes reactions between water and olefins to form alcohols, such as an acidic catalyst. Acid catalysts can be chosen from among those known to those skilled in the art including supported acids such as sulfated zirconia, sulfated silica, or sulfated alumina, or solid acids such as solid phosphoric acid, zeolites, pillared clays, or amorphous silica-alumina mixtures. Preferred catalysts are sulfated silica, sulfated alumina, sulfated zirconia, solid phosphoric acid, or acid-treated clays. Temperatures are preferably between 170 and 400° C., more preferably 220 to 300° C. in the zone holding the catalyst.

In another embodiment, the olefin-containing stream is mixed with methanol (typically in the vapor phase) and passed over a catalyst to form an ether before being fed to the pyrolysis reactor. Temperatures in the ether-forming stage are preferably between 200 and 400° C. in the zone holding the catalyst. The catalyst is a catalyst that catalyzes reactions between alcohols and olefins to form ethers, such as an acidic catalyst, preferably a solid acid catalyst. Acid catalysts can be chosen from among those known to those skilled in the art including supported acids such as sulfated zirconia, sulfated silica, or sulfated alumina, or solid acids such as solid phosphoric acid, zeolites, pillared clays, or amorphous silica-alumina mixtures. Preferred catalysts are sulfated silica, sulfated alumina, sulfated zirconia, solid phosphoric acid, or acid-treated clays. Ion exchange resins in which an acidic group such as sulfate, nitrate, phosphate, carboxylate, benzoate, or trifluoroacetate is attached to a polymeric backbone, such as Amberlyst, can also be used. Temperatures are preferably between 170 and 400° C., more preferably 220 to 300° C. in the zone holding the catalyst. Pressure for the reaction is preferably at least 1 atm, and in some embodiments in the pressure range of 1 to 50 atm. The molar ratio of alcohol-to-olefin is preferably in the range of 0.1 to 10, more preferably 0.5 to 5, and in some embodiments 0.5 to 2. In some preferred embodiments, the methanol or other alcohol reactant is obtained either directly or indirectly from the pyrolysis of biomass.

The olefins can also be reacted with higher alcohols, such as ethanol, propanol (n-propanol, iso-propanol, or mixtures thereof) or mixtures of alcohols, to form ethers. This reaction can be conducted in the presence of catalysts and under the conditions mentioned above with respect to methanol. A more preferred temperature range is 100 to 200° C. In addition or as an alternative to the catalysts mentioned above for methanol, zeolite catalysts can be present in the reactor in which ethers are produced. Examples of suitable zeolites include: zeolite Y, zeolite X, ZSM-3, ZSM-5, ZSM-12, ZSM-20, ZSM-23, ZSM-35, ZSM-38, ZSM-50, MCM-22, and mixtures thereof.

In a related variation, the ethers can be produced in two stages, a first stage in which the olefin(s) and alcohol(s) are reacted in the presence of a zeolite, and a second stage in which the unreacted olefins are reacted with alcohol in the presence of a resin-based catalyst, such as Amberlyst 15. The two-stage reaction provides the advantage of protecting the resin-based catalyst from the initial process conditions, such as relatively higher temperatures and/or impurities in the olefin-containing stream that could degrade the resin-based catalyst. Thus, there could be a separation process that is conducted between stages. Typically, the two stages are conducted in separate reactors. In some preferred embodiments, the zeolite catalyst from the first stage is passed into an FCP reactor and/or passed into a regeneration stage that is shared with catalyst from the FCP reactor.

In some embodiments, the olefin-containing stream is treated in the presence of a catalyst and is then fed to an FCP reactor (typically the same FCP in which the original products were formed). The catalyst used in the olefin treatment stage may be carried along with a product stream (i.e., a product stream resulting from treatment of the olefin stream) into a FCP reactor. Thus, the FCP reactor can contain at least two catalysts, one of which is preferred for the catalytic pyrolysis and one of which is preferred for the olefin conditioning. In some embodiments of this invention a catalyst that is used for the olefin conditioning is the same as, or similar to, a catalyst used in the FCP reactor. The catalyst used in the olefin conditioning can be fed to the pyrolysis reactor in parallel with a pyrolysis catalyst (which is typically a recycled catalyst). In some preferred embodiments, the olefin-treatment catalyst enters the reactor is then regenerated in a regenerator along with a pyrolysis catalyst. If the catalysts are not identical, they can either be separated, or used without separation in either or both of the FCP reactor and/or an olefin conditioning reactor.

In any of the processes described herein, the olefin-containing gas or the treated stream can be partially separated into different fractions for functionalization to remove non-reactive components or to purge excess materials.

In some embodiments the olefin-containing stream can be hydrogenated by treatment with hydrogen to at least partially reduce the olefin or alkyne content. In some preferred embodiments the olefin-containing stream is treated with hydrogen in the presence of a hydrogenation catalyst. Typical olefin or alkyne hydrogenation catalysts comprise Pd, Pt, Ag, Ni, Rh, Ru, Cr, or some combination of these on an oxide or other inert support. The hydrogenation of alkynes can be used in combination with other olefin-conditioning steps. Preferably the hydrogenation step would be carried out before other olefin-conditioning steps.

What is claimed:

1. A method for producing one or more fluid hydrocarbon products from a hydrocarbonaceous material, comprising:
feeding a hydrocarbonaceous material is fed to a reactor;
pyrolyzing at least a portion of the hydrocarbonaceous material within the reactor under reaction conditions sufficient to produce one or more pyrolysis products;
separating the products into at least a first fraction and a second fraction, where the first fraction comprises at least one olefin;
treating the first fraction by adding a chemical compound and/or reacting at least some of the olefins in the first fraction to produce a treated fraction; and
adding the treated fraction to a pyrolysis reactor.

2. The method of claim 1 wherein the treated fraction is recycled to the same reactor that produced the pyrolysis products.

3. The method of claim 1 wherein the step of treating comprises one or any combination of the following steps: conversion of olefins to alcohols or ethers; addition of radical inhibiting agents; low temperature (80-400° C.) polymerization of olefins;
reaction with CO to form carboxylic acids; alkylation to form alkylated aromatics, and hydrogenation of alkynes to olefins.

4. The method of claim 1 wherein a gaseous product stream from the reactor is separated into an olefin poor and olefin rich stream, and at least a portion of the olefin rich stream coming from an olefins separator is treated before mixing with a full recycle gas stream, thereby reducing the volume to be treated.

5. The method of claim 1 wherein one or more fluid hydrocarbon products are produced from the pyrolysis products by a treatment selected from the group consisting of dehydration, decarbonylation, decarboxylation, isomerization, oligomerization, and dehydrogenation reactions.

6. The method of claim 1 wherein at least a portion of the olefins produced by the pyrolysis reaction and/or a subsequent reaction are treated and recycled into a feed stream comprising the hydrocarbonaceous biomass material.

7. The method of claim 1 wherein the step of treating comprising one or more of the following reactions: hydrogenation, hydrolysis, hydroformylation, cyclization, dimerization, and polymerization.

8. The method of claim 1 wherein the step of pyrolyzing is fast catalyzed pyrolysis and wherein the treating step is conducted at a temperature that is lower than the fast catalyzed pyrolysis in a secondary reactor.

9. The method of claim 1 wherein the step of treating comprises contacting the first fraction with an acid catalyst at conditions to cause dimerization or oligomerization.

10. The method of claim 9 wherein the first fraction comprises ethylene and propylene and wherein the step of treating consumes more of the propylene than the ethylene.

11. The method of claim 1 wherein the first fraction is contacted with a used fast pyrolysis catalyst that is partially deactivated or coked.

12. The method of claim 1 wherein the reactor is a FCP reactor and wherein the first fraction comprises ethylene and the step of treating comprises conversion to propylene prior to recycle to the FCP reactor.

13. The method of claim 1 wherein a radical inhibitor is added to the first fraction or to the treated fraction before the treated fraction is added to the pyrolysis reactor.

14. The method of claim 1 wherein the first fraction is treated by alkylation wherein the first fraction is mixed with an aromatic-containing stream and contacted with an acid catalyst.

15. The method of claim 1 wherein the first fraction is mixed with water vapor and passed over a hydrolysis catalyst before being fed to the pyrolysis reactor.

16. The method of claim 1 wherein the first fraction is reacted with an alcohol to produce an ether.

17. The method of claim 16 wherein the molar ratio of alcohol to olefin in in the range of 0.1 to 10.

18. The method of claim 16 comprising a first stage where the alcohol and olefin are reaction in the presence of a zeolite and a second stage in which unreacted olefins are reacted with alcohol in the presence of a resin-based catalyst.

19. The method of claim 1 wherein a catalyst used in the treating step is fed into the pyrolysis reactor in parallel with a pyrolysis catalyst.
